(19) United States
(12) Patent Application Publication
(10) Pub. No.: US 2004/0146486 A1
(43) Pub. Date: Jul. 29, 2004

(54) Hybrid vector system for use as a vaccine

(76) Inventor: Juan Sun, Aurora, CO (US)

Correspondence Address: HOGAN & HARTSON LLP, ONE TABOR CENTER, SUITE 1500, 1200 SEVENTEENTH ST, DENVER, CO, 80202, US

(21) Appl. No.: 10/350,791

(22) Filed: Jan. 24, 2003

(51) Int. Cl. A61K 48/00; A61K 39/12; A61K 39/02

(52) U.S. Cl. 424/93.2; 424/85.1; 424/85.2; 424/199.1; 424/200.1

(57) ABSTRACT

A gene delivery system is composed of a yeast hybrid delivery system vector. Two gene-expressing units form the yeast hybrid vaccine delivery system by gene type mating. One unit is engineered to constitutively express a co-stimulator and another can be engineered to express single, fusion or multiple antigens derived from disease causing pathogen. This yeast hybrid vaccine easy delivery system vector is designed to develop novel vaccines. The candidate vaccines include AIDs, cancer, Hepatitis C, Parainfluenza, malaria, autoimmunity and other infectious diseases.

This study was supported by the Chinese Natural Scientific Foundation Grant No. 39800162 to Juan Sun.

### BACKGROUND OF THE INVENTION

1. Field of the Invention

The present invention relates generally to compositions and methods for treating and/or preventing chronic infectious diseases, cancers, and autoimmune diseases. More specifically, the present invention relates to an expression vector comprising at least two polypeptide units wherein the first polypeptide unit expresses a co-stimulator and the remaining polypeptide unit(s) expresses single, fusion, or multiple antigens derived from the disease causing agent and methods of using the expression vector in vaccination, tumor therapy, and prophylaxis.

2. Description of the State of Art

Along with water sanitation, prevention of infectious diseases by vaccination is the most efficient, cost-effective, and practical method of disease prevention. No other modality, not even antibiotics, has had such a major effect on mortality reduction and population growth. The impact of vaccination on the health of the world's people is hard to exaggerate. Vaccination, at least in parts of the world, has controlled the following nine major diseases: smallpox, diphtheria, tetanus, yellow fever, pertussis, poliomyelitis, measles, mumps and rubella. In the case of smallpox, the disease has been totally eradicated from the world. The effectiveness of a vaccine depends upon its ability to elicit a protective immune response, which will be generally described below.

The means by which vertebrates, particularly birds and mammals, overcome microbial pathogenesis is complex. Pathogens that invade a host provoke a number of highly versatile and protective systems. If the microbial pathogen or its toxins successfully penetrate the body's outer defenses and reach the bloodstream, then the lymphoid tissue of the spleen, liver, and bone marrow will remove and destroy the foreign material as the blood circulates through these organs. Lymphoid tissue is composed primarily of a meshwork of interlocking reticular cells and fibers. Clinging to the interstices of the tissues are large numbers of leukocytes, more specifically, lymphocyte cells, and other cells in various stages of differentiation, such as plasma cells, lymphoblasts, monocyte-macrophages, eosinophils and mast cells. The two main lymphocytes, T cells and B cells, have different and complementary roles in the mediation of the antigen-specific immure response.

The immune response is an exceedingly complex and valuable homeostatic mechanism that has the ability to recognize foreign pathogens. The initial response to foreign pathogen is called “innate immunity” and is characterized by the rapid migration of natural killer cells, macrophages, neutrophils, and other leukocytes to the site of the foreign pathogen. These cells can either phagocytose, digest, lyse, or secrete cytokines that lyse the pathogen in a short period of time. The innate immune response is not antigen-specific and is generally regarded as a first line of defense against foreign pathogens until the “adaptive immune response” can be generated. Both T cells and B cells participate in the adaptive immune response. A variety of mechanisms are involved in generating the adaptive immune response. A discussion of all the possible mechanisms of generating the adaptive immune response is beyond the scope of this section; however, some mechanisms which have been well-characterized include B cell recognition of antigen and subsequent activation to secrete antigen-specific antibodies and T cell activation by binding to antigen presenting cells.

B cell recognition involves the binding of antigen, such as bacterial cell wall, bacterial toxin, or a glyco-protein found on a viral membrane to the surface immunoglobulin receptors on B cells. The receptor binding transmits a signal to the interior of the B cell. This is what is commonly referred in the art as “first signal.” In some cases, only one signal is needed to activate the B cells. These antigens that can activate B cells without having to rely on T cell help are commonly referred to as T-independent antigens (or thymus-independent antigens). In other cases, a “second signal” is required and this is usually provided by T helper cells binding to the B cell. When T cell help is required for the activation of the B cell to a particular antigen, the antigen is then referred to as T-dependent antigen (or thymus-dependent antigen). In addition to binding to the surface receptors on the B cells, the antigen can also be internalized by the B cell and then digested into smaller fragment within the B cell and presented on the surface of B cells in the context of antigenic peptide-MHC class II molecules. These peptide-MHC class II molecules are recognized by T helper cells that bind to the B cell to provide the “second signal” needed for some antigens. Once the B cell has been activated, the B cells begin to secrete antibodies to the antigen that will eventually lead to the inactivation of the antigen. Another way for B cells to be activated is by contact with follicular dendritic cells (FDCs) within germinal centers of lymph nodes and spleen. The follicular dendritic cells trap antigen-antibody (Ag-Ab) complexes that circulate through the lymph node and spleen and the FDCs present these to B cells to activate them.

Another well-characterized mechanism of adaptive immune response to antigens is the activation of T cells by binding to antigen presenting cells such as macrophages and dendritic cells. Macrophages and dendritic cells are potent antigen presenting cells. Macrophages have a variety of receptors that recognize microbial constituents such as macrophage mannose receptor and the scavenger receptor. These receptors bind microorganisms and the macrophage engulfs them and degrades the microorganisms in the endosomes and lysosomes. Some microorganisms are destroyed directly this way. Other microorganisms are digested into small peptides that are then presented to T cells on the surface of the macrophages in the context of MHC class II-peptide complexes. T cells that bind to these complexes become activated. Dendritic cells are also potent antigen presenting cells and present peptide-MHC class I molecules and peptide-MHC class II molecules to activate T cells.

When a B cell binds to an antigen which has never been encountered, the cell undergoes a developmental pathway called “isotype switching”. During the developmental changes, the plasma cells switch from producing general IgM type antibodies to producing highly specific IgG type antibodies. Within this population of cells, some undergo repeated divisions in a process called “clonal expansion”. These cells mature to become antibody factories that release immunoglobulins into the blood. When they are fully mature, they become identified as plasma cells, cells that are capable of releasing about 2,000 identical antibody molecules per second until they die, generally within 2 or 3 days after reaching maturity. Other cells within this group of clones never produce antibodies but function as memory cells that will recognize and bind that particular antigen upon encountering the antigen.

As a consequence of the initial challenge by an antigen there are now many more cells identical to the original B cell or parent cell, each of which is able to respond in the same way to the antigen as the original B cell. Consequently, if the antigen appears a second time, it will encounter one of the correct B cells sooner, and since these B cells are programmed for the specific IgG antibody, the immune response will begin sooner, accelerate faster, be more specific and produce greater numbers of antibodies. This event is considered a secondary or anamnestic response. Immunity can persist for years because memory cells survive for months or years and also because the foreign material is sometimes reintroduced in minute doses that are sufficient to constantly trigger low-level immune responses. In this way the memory cells are periodically replenished.

Following the first exposure to an antigen the response is often slow to yield antibody and the amount of antibody produced is small, i.e., the primary response. On secondary challenge with the same antigen, the response, i.e., the secondary response, is more rapid and of greater magnitude thereby achieving an immune state equal to the accelerated secondary response following re-infection with a pathogenic microorganism, which is the goal that is sought to be induced by vaccines.

Classically, active vaccines have divided into two general classes: subunit vaccines and whole organism vaccines. Subunit vaccines are prepared from components of the whole organism and are usually developed in order to avoid the use of live organisms that may cause disease or to avoid the toxic components present in whole organism vaccines. The use of purified capsular polysaccharide material of H. influenza type b as a vaccine against the meningitis caused by this organism in humans is an example of a vaccine based upon an antigenic component. Whole organism vaccines, on the other hand, make use of the entire organism for vaccination. The organism may be killed or alive (usually attenuated) depending upon the requirements to elicit protective immunity. The pertussis vaccine, for example, is a killed whole cell vaccine prepared by treatment of Bordetella pertussis cells with formaldehyde. The use of killed cells, however, is usually accompanied by an attendant loss of immunogenic potential, since the process of killing usually destroys or alters many of the surface antigenic determinants necessary for induction of specific antibodies in the host.

In marked contrast to killed vaccines live attenuated vaccines are comprised of living organisms that are benign but typically can replicate in a host tissues and presumably express many natural target immunogens that are processed and presented to the immune system similar to a natural infection. This interaction elicits a protective response as if the immunized individual had been previously exposed to the disease. Most of the work defining attenuating mutations for the construction of live bacterial vaccines has been performed in S. spp. since they establish an infection by direct interaction with the gut associated lymphoid tissue (GALT), resulting in a strong humoral immune response. They also invade host cells and thus are capable of eliciting a strong cell mediated response. Eisenstein, Intracellular Bacterial Vaccine Vectors (Paterson, ed., Wiley-Liss, Inc.) pp. 51-109 (1999); Hone, et al. Intracellular Bacterial Vaccine Vectors (Paterson, ed., Wiley-Liss, Inc.) pp. 171-221 (1999); Sirard, et al., Immun. Rev., 171:5-26 (1999). Ideally, these attenuated microorganisms maintain the full integrity of cell-surface constituents necessary for specific antibody induction yet are unable to cause disease, because, for example, they fail to produce virulence factors, grow too slowly, or do not grow at all in the host. Additionally, these attenuated strains should have substantially no probability of reverting to a virulent wild-type strain. Traditionally, live vaccines have been obtained by either isolating an antigenically related virus from another species, by selecting attenuation through passage and adaptation in a nontargeted species or in tissue cultures, or by selection of temperature-sensitive variants. The first approach was that used by Edward Jenner who used a bovine pox-virus to vaccinate humans against smallpox.

Classic vaccine theory implies that prophylactic inoculation with a non-lethal or attenuated pathogen will evoke an immune response capable of providing protection against infection with the same or similar pathogens on subsequent encounter. Such an approach is feasible with viruses, and to a lesser extent with bacteria, which possess a defined number of antigens. However, this is not the case with tumour cells, which may express a limitless number of antigens. In addition, unlike classical vaccine strategies, anticancer vaccines must induce an immune response after antigen exposure rather than before it. If anticancer vaccines are to be successful they must induce an immune response capable of eradicating existing disease, which will require a greater understanding of the nature of tumour antigens and of host-tumour interactions. Current vaccine concepts have been directed toward the induction of cellular immunity.

As yet, no vaccine has been effective in conferring protection against HIV infection. Attempts to develop vaccines have thus far failed. Certain antibodies reactive with HIV, notably anti-GP160/120 are present at high levels throughout both the asymptomatic and symptomatic phases of the HIV infection, suggesting that rather than playing a protective role, such antibodies may in fact promote the attachment and penetration of the virus into the host cell. More significantly, current vaccines do not induce efficient cellular responses against the infected cells, the source of newly released virions.

Although there is considerable evidence from scientific and clinical studies that the immune system is capable of destroying cancerous tissue, in most cases the immune system either fails to recognize the tumor or the response that is generated is too weak to be effective. See, Farzaneh, et al., Immunol. Today, 19:294 (1998). While early detection may cure tumors in many cases, once the disease becomes metastatic to distant organs, it is almost always fatal. Furthermore, the disappointing results observed with chemotherapy, radiotherapy and surgery, individually or in combination, has shifted the attention of many investigators to immunological or biological agents. See, Ockert, et al., Immunol. Today, 20:63 (1999). As such, increasing the capacity of the immune system to mediate tumor regression has been a major goal in tumor immunology. Progress towards this goal has recently been aided by the identification of immunogenic tumor antigens and by a better understanding of the mechanisms of T cell-mediated immune response and tumor escape. See, Boon, et al., Immunol. Today, 18:267 (1998); Chen, Immunol. Today, 19:27 (1998).

An understanding of the mechanisms by which some animals reject tumors whereas others display progressive tumor outgrowth is gradually evolving based on an appreciation of the underlying concepts of cellular and tumor immunology. Simply put, these are that tumor cells can be eliminated by the immune system and that cellular cytotoxicity plays a major role in antitumor immunity and the effector cells in many cases are either CD8+CTL or CD4⁺ Th cells. See, Denfeld, et al., Int J. Cancer, 62:259; Greten, et al., J. Clin. Oncol., 17:1047 (1998); Sampson, et al., Proc. Natl. Acad. Sci. USA, 93:10399 (1996). However, the induction and amplification of an effective T cell-mediated immune response in malignancies characterized by poor immungenicity is the most challenging aspects of tumor vaccine development (Sampson, et al., 1996). A two signal model of lymphocyte activation postulates that for optimal activation, lymphocytes require both an antigen-specific signal delivered through TCR and an antigen-nonspecific costimulatory signal. See, van Seventer, et al., Curr. Opin. Immunol., 3:294 (1991); Linsley, et al., J. Exp. Med., 173:721 (1991). In this regard, tumor cells may effectively evade the immune system by several mechanisms which are not only confined to tumor cells, but may also be related to impaired function of the immune response in a tumor bearing host (Gretten and Jaffee, 1999). These include: defective expression of MHC complex on tumor cells, antigen processing defects, lack of T cell recognition by outgrowth of antigen negative clones of tumor cells, inadequate expression of costimulatory molecules on tumor cells, inadequate expression of adhesion molecules on tumor cells, inadequate expression of Fas receptor and/or FaL expression on tumor cells, immune-suppressive cytokine secretion into tumor microenvironment, and host defense failure due to impaired immune cell function (Boon, et al., 1997).

Therefore, in the majority of cases the immune system either fails to recognize the tumor or the response that is generated is too weak to be effective. Furthermore, the management of residual and metastatic disease is a central problem in the treatment of tumors. During a normal immune response, full activation of antigen-specific naive T cells requires at least two distinct signals from surface receptors to proliferate in response to antigens. See, Young, et al., J. Clin. Invest., 90:229 (1992); Allison, et al., Science, 270:932 (1995). One of the signals is supplied by T cell receptor (TCR) engagement with peptide (antigen)-loaded major histocompatibility complex (MHC) molecules on antigen-presenting cells (APC). The second signal, at present poorly understood, can be delivered by the interaction of various molecules on the surface of T cells and the APC, one of which is the interaction of CD28 and B7-1. See, Linsley, et al., 1991; Young, et al., 1992; Bluestone, Immunity, 2:555 (1995). The combination of these two signals leads to activation, clonal expansion and differentiation into effector cells of T lymphocytes. See, Guerder, et al., J. Immunol., 155:5167 (1995); Webb, et al., Blood, 86:3479 (1995); Thompson, Cell, 81:979(1995). Effector T lymphocytes, unlike naive T cells, no longer require co-stimulatory signals to recognize and kill antigen-bearing targets. After the immune response, a fraction of the effector cells remain as memory cells that form the basis of a faster and stronger immune response upon subsequent presentation of the same antigen (Gray, Ann. Rev. Immunol., 11:49 (1993); Ahmed, et al., Science, 272:54 (1996). The absence of second signal results in T cell clonal anergy, thus preventing clonal expansion of T lymphocytes. See, Chen, (1998); van Gool, et al., Res. Immunol., 146:183 (1994).

Although many tumor cells express target antigens, they are generally incapable of stimulating an immune response. See, Boon, et al., (1997); Boon, et al., J. Exp. Med., 183:725 (1996). Cytotoxic T-lymphocytes (CTL) have been recognized as a critical component of the immune response to tumors. See, Boon, et al., (1996); Chen, et al., J. Exp. Med., 179:523 (1994). CTL responses are sufficient to protect against tumors and can eliminate even established cancers in murine models. See, Mogi, et al., Clin. Cancer Res., 4:713 (1998) and in humans, see, Gong, et al., Proc. Natl. Acad. Sci. USA, 97:2715 (2000). Inducing strong antigen-specific CTL responses is the goal of many current cancer vaccine strategies.

The development of CTL-dependent anti-tumor immunization strategies depends on both the identification of tumor antigens recognized by CTLs and the development of methods for effective antigen delivery. CTL target tumors through recognition of a ligand consisting of a self MHC class I molecule and a peptide antigen generally derived from proteins synthesized within the tumor cell. However, for CTL induction and expansion to occur, the antigenic ligand must be presented to CTLs in the appropriate context of co-stimulation usually provided by professional APCs. Delivery of exogenous antigen to the endogenous MHC class I restricted processing pathway of professional APCs is a critical challenge in cancer vaccine design. Antigen delivery strategies currently under development include immunization with defined peptides, particulate proteins capable of accessing the class I pathway of professional APCs in vivo, heat shock proteins isolated from tumor cells, or adoptive transfer of antigen-loaded APCs. In addition, recent studies suggest that DNA vaccines encoding tumor antigens delivered by viral vectors or liposomes, or as naked DNA, can induce potent anti-tumor immunity.

In addition to the challenge of antigen delivery, most current tumor immunization strategies depend on the identification and production of appropriate tumor antigens. To overcome this limitation, tumor cells themselves may be used as immunogens as described in ACV (autologous cell vaccine). Engineering tumor cells to provide APC function could potentially result in polyvalent immunization to multiple tumor-specific epitopes, while obviating the need to identify specific tumor antigens. Many tumor vaccine strategies, including cytokine-transduced tumor cells, commonly referred to as gene therapy. See, Asher, et al., J. Immunol., 146:3227 (1991); Tahara, et al., Ann. NY Acad. Sci., 795:275 (1996); Lotze, et al., Ann. NY Acad. Sci., 795:440 (1996); Rakhmilevich, et al., Hum. Gene Ther., 8:1301 (1997); Nawrocki, et al., Cancer Treat Rev., 25:29 (1998), synthetic peptide vaccine (Rosenberg, et al., Nature Med., 4:321 (1998), tumor-antigen (peptide)-pulsed dendritic cells (Flamand, et al., Eur. J. Immunol., 24:605 (1994); Bianchi, et al., J. Immunol., 157:1589 (1996); Ashley, et al., J. Exp. Med., 186:1177 (1997); Yang, et al., Cell. Immunol., 179:84 (1997); Thurner, et al., J. Exp. Med., 190:1669 (1999), and DNA vaccine (Leclerc, et al., Immunol. Today, 19:300 (1998); Akbari, et al., J. Exp. Med., 189:169 (1999) are currently under pre-clinical and clinical investigation but to date have yielded only marginal immunological and clinical response.

As discussed above, methods requiring administration of peptides or proteins have inherent limitations, due to turn-over and degradation. Furthermore, generation of CTLs from CTL precursors (CTL-Ps) appears to require the interaction of IL-2 with high-affinity IL-2 receptor, resulting in proliferation and differentiation of the antigen-activated CTL-P into an effector CTL. Inadequacy of IL-2 induces Th1 cells and CTLs to undergo programmed cell death by apoptosis. In this way, the immune response is rapidly terminated, lessening the likelihood of nonspecific tissue damage from the inflammatory response.

In order to overcome the limitations of current CTLs approach for HIV and cancers, there is an urgent need for the development of new and improved vaccine delivery systems. A likely ideal component of new and improved vaccines will be more potent vaccine adjuvants. The adjuvants to be used in these vaccines may have to closely mimic an infection and/or induce localized tissue damage to elicit protective immunity. This may be achieved through the use of particular delivery systems, which have similar compatibilities to pathogens and are able to target antigens to macrophages and DC. In addition, it may also be necessary to deliver one or more pathogen associated molecular patterns (PAMP), which will more fully activate the innate response and may result in the desired type of adaptive response. Furthermore, a delicate balance may be achieved between the two-signal model of T cell activation and long-term maintain of the memory T cell activity. Many of these new generation vaccines will require the induction of potent cell-mediated Immunity (CM1), including CTL responses. Accumulated research shows that induction of CTL is difficult With proteins and may require much stronger stimulation of the immune system than is normally required for a humoral response. Therefore, DNA remains an attractive approach for many pathogens, but needs to be deliverd more effectively to improve the potency in human. A state-of-the-art vaccine delivery system, yeast hybrid vaccine, has been constructed to exert strong, long-term CTLs memory for invoking MHC class I and II dependent protective immunity. It is a mean to promote uptake of the delivery system by the relevant cells for optimal efficacy.

### SUMMARY OF THE INVENTION

According to a first aspect of the invention, there is provided a gene delivery system comprising a DNA molecule encoding a co-stimulator, such as a cytokine operably linked to a promoter and a DNA molecule encoding at least one antigen operably linked to a promoter.

According to a second aspect of the invention, there is provided a method of eliciting an anti-tumor immune response in a patient comprising: isolating cancerous cells from a patient; transfecting cancerous cells cancerous cells with a gene delivery system comprising a DNA molecule encoding a co-stimulator, such as IL-2 and a DNA molecule encoding at least one antigen operably linked to a promoter capable of directing expression of the DNA molecule in said cancerous cells; incubating said transfected cells under conditions whereby the co-stimulator and the antigen(s) are expressed; and eliciting an anti-tumor immune response in the patient by injecting said transfected cells into the patient.

According to a third aspect of the invention there is provided a method of eliciting a cytotoxic T-lymphocyte (CTL) response against a pathogenic microorganism such as a virus.

According to a fourth aspect of the invention, there is provided an expression system comprising a DNA molecule encoding IL-2 and an antigen each operably linked to independent promoters.

According to a fifth aspect of the invention there is provided a method of eliciting an immune response in a vertebrate comprising introducing to the vertebrate the gene delivery system of the present invention encoding for the antigen to which an immune response is desired.

### BRIEF DESCRIPTION OF THE DRAWINGS

The accompanying drawings, which are incorporated in and form a part of the specification, illustrate the preferred embodiment of the present invention, and together with the description serve to explain the principles of the invention.

In the Drawings:

FIG. 1 is a diagrammatic representation of the operating principal of the present invention.

FIG. 2 is a diagrammatic representation of one embodiment of a DNA vector of the present invention.

FIG. 3 shows a graph comparing the expression of levels of hIL-2.

FIG. 4 is a bar graph demonstrating cytotoxic T lymphocyte activity in mice vaccinated with indicated vaccines.

FIG. 5 are photographs of mice treated with either PB5 or J6V factor.

### DETAILED DESCRIPTION OF INVENTION AND PREFERRED EMBODIMENTS

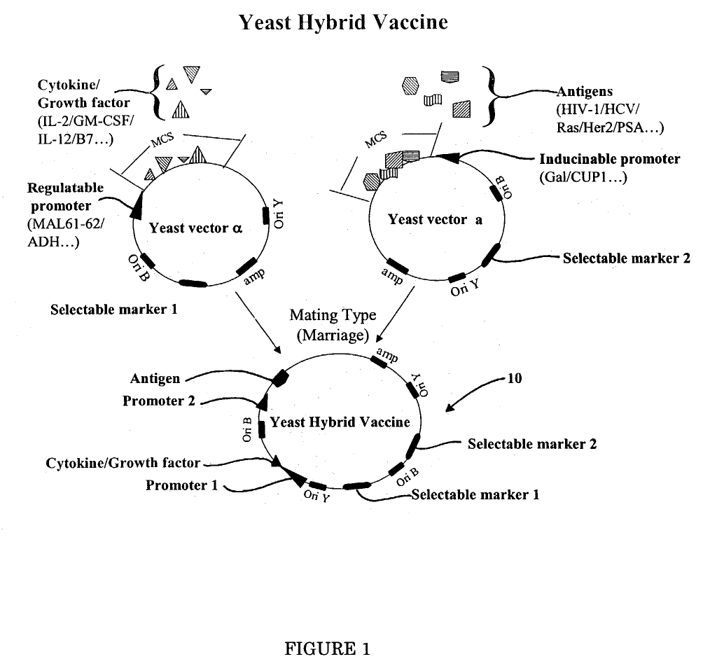

The gene-delivery system of the present invention according to this invention is capable of exerting strong, long-term cytotoxic T-lymphocyte (CTL) memory for invoking major histocompatability (MHC) class I and II dependent protective immunity. Plasmid or expression vector, shown in FIG. 1, provides several significant functions for purposes of this invention. Plasmid 10 provides a method for introducing and expressing, in independent fashion, both an antigen gene or genes and a co-stimulator. Thus every antigen expressed is capable of having its perfect partner to co-stimulate the protective immunity. It is very likely that the antigen could be less reactogenic than with its co-stimulator delivered coincidentally. Examples of antigen are, but are not limited to, portions of viral, bacterial, parasitic and cancer cells in single, multiple or combinational fashion, such as Fragment C of tetanus toxin, the B subunit of cholera toxin, the hepatitis B surface antigen, Vibrio cholerae LPS, HIV antigens and/or Shigella soneii LPS. Other antigens may be fungal antigens, protozoan antigens, helminth antigens, ectoparasite antigens, and cancer antigens. Cancer antigens, such as but not limited to K-ras, EGFR, HER2, PSMA, CEA, MAGE, MART-1 may also be used. Examples of co-stimulators are, but are not limited to, cytokine, such as but not limited to IL-2, TNF, IFN, B7-1, GM-CSF, IL-12, IL-3, IL-4, IL-5, IL-6, IL-10, IL-14, IL-14, IL-18, B7-2, CD28, CD40, TNFR, lylphotoxin-betaR, NF-KappaB, ICAM-1, LFA-3, M-CSFR, mM-CSF, Flt3-L, SCF, TPO, CD80 and CD58.

Essentially, the gene delivery system or plasmid 10 of the present invention, as shown in FIG. 1, originates with two separate plasmids that are mated by cross gene-type mating using a eukaryotic organism such as but not limited to yeast, such as Saccharomyces, Schizosaccharomyces, Yarrowia, Candida, Hansenula. Alternatively, the gene delivery system of the present invention can be formed using prokaryotic organisms also, such as but not limited to, E. coli, and viral plasmid. Thus whether a eukaryotic organism or prokaryotic organism is utilized a final product that comprises a coding sequence for a co-stimulator under the control of a promoter that may be inducible or consitutive, and a first polylinker multiple cloning site for insertion of various antigens under the control of a promoter that may also be inducible or constitutive is produced. A sequence of nucleotides adapted for directional ligation, i.e., a polylinker, is a region of the DNA expression vector that (1) operatively links for replication and transport the upstream and downstream translatable DNA sequences, and (2) provides a site for directional ligation of a DNA sequence into the vector. Typically, a directional polylinker is a sequence of nucleotides that defines two or more restriction endonuclease recognition sequences. Upon restriction cleavage, the two sites yield cohesive termini to which a translatable DNA sequence can be ligated to the DNA expression vector. Preferably, the two restriction sites provide, upon restriction cleavage, cohesive termini that are non-complementary and thereby permit directional insertion of a translatable DNA sequence into the cassette. Where the sequence of nucleotides adapted for directional ligation defines numerous restriction sites, it is referred to as a multiple cloning site. Additionally, the vector may contain a second polylinker multiple cloning site for insertion of selectable marker genes, an origin of replication, and a phenotypically selectable marker gene to identify host cells which contain the expression vector. Examples of markers typically used in eukaryotic expression vectors include neo, leu2, ura3, trp1, ade1, his3. The gene delivery system of the present invention is then administered into the host in need of treatment.

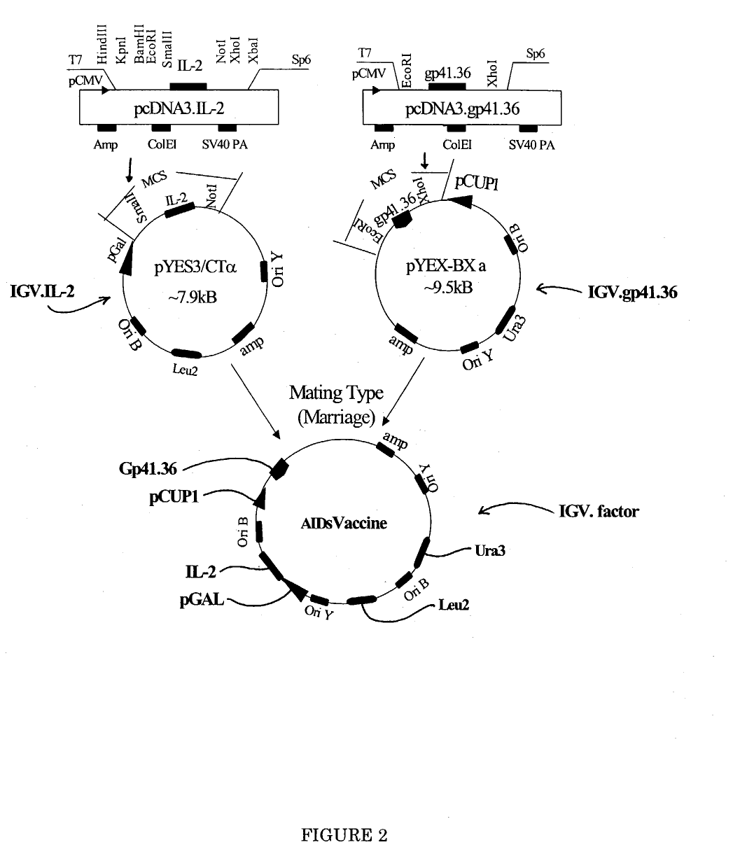

The gene delivery system of the present invention, described above, is constructed as shown diagrammatically in FIG. 1. First, a plasmid having a functional polynucleotide that expresses a co-stimulator, discussed above, is constructed. Other features on this plasmid are shown in FIG. 2. Preferably, the promoter region of the functional polynucleotide, is the promoter region of a eukaryotic organism such as but not limited to yeast Sccharomyces cerevisine or pathogenic yeast such as Candida albicans. Promoters identified by the method of the invention can be inducible or constitutive promoters. Inducible promoters can be regulated, for example, by nutrients (e.g., carbon sources, nitrogen sources, and others), drugs (e.g., drug resistance), environmental agents that are specific for the infection process (e.g., serum response), and temperature (e.g., heat shock, cold shock). A second plasmid having a multiple cloning site under the regulatory control of a promoter region is constructed for insertion of the desired antigen. The resulting plasmids are then transformed individually into two separate eukaryotic organisms such as haploid yeast strains. Where the host cell is eukaryotic, various methods of DNA transfer can be used. These include transfection of DNA by calcium phosphate-precipitates, conventional mechanical procedures such as microinjection, insertion of a plasmid encased in liposomes, spheroplast, electroporation, salt mediated transformation of unicellular organisms, or the use of viral vectors. A library of host cells, wherein each host cell contains a vector according to the description above, is also included in the invention.

Alternatively, the expression vector 10 may be constructed using prokaryotic promoters in the event a prokaryotic organism is used as the host organism for delivering expression vector 10 to the vertebrate for which an immune response is being elicited. Where the host organism is a prokaryotic organism expression vector 10 may be introduced into the host strain through a number of well known methods such as, transduction, conjugation, transformation, electroporation, transfection, etc.

When yeast are used as the host organism, once the respective plasmid has been successfully transformed into the respective organism, the transformants are selected with an amino acid drop out media and then mated together by cross-gene type mating. See, Guthrie Fink, Guide to Yeast Genetics & Molecular Biology, Methods in Enzymology, 194:3-231 (1999). The mated plasmid 10 is able to express both the co-stimulator and the antigen in independent fashion. The final strain is selected in the double amino acid (one from each strain) drop out selection media. In the event yeast are not used as the host organism successful identification of transformants having expression vector 10 is achieved by simply selecting for the presence of the desired selectable markers.

The present invention may be used for both human or veterinary vaccines as a therapeutic and/or prophylactic against for many disease states such as but not limited to, influenza, leukemia, HIV, hepatitis C, hepatitis B, human papilloma virus, genital herpes, autoimmune diseases, malaria, lung cancer, liver cancer, prostate cancer, ovarian cancer, cervical cancer, therapeutic rheumatoid arthritis, and other bacterial, viral, parasitic infectious diseases.

In one embodiment the method of use of the vaccine comprises isolating macrophages from donors or the patient or be treated. The isolated macrophages are transfected with the vector of the present invention and are growth under conditions such that the co-stimulatory molecule and antigen are expressed.

In order to construct the lung cancer vaccine, the granulocyte-macrophage colony stimulator factor (GM-CSF) is engineered on yeast vector a under the control of a constitutive yeast promoter ADC. Lung cancer antigen, epidermal growth factor receptor (EGFR) is engineered on the yeast vector a. The resulting haploid yeast plasmids are transformed to yeast host strains W303 α and a respectively. The two selected transformants are mated by gene-typing cross mating and selected by leu2/ura3 double amino acid dropout selection. The resulting diploid transformant becomes the final lung cancer vaccine product.

The vaccines of the present invention are typically administered parenterally, by injection for example, either subcutaneously, intramuscularly, intraperitoneally or intradermally. Administration can also be intranasal, intrapulmonary (i.e., by aerosol), oral and intravenous. Additional formulations which are suitable for other modes of administration include suppositories and, in some cases, oral formulations. The route of administration will depend upon the condition of the individual and the desired clinical effect. For administration to farm animals, such as chickens, preferred administration are oral formulations. The vaccines are suitable for systemic administration to individuals in unit dosage forms, sterile parenteral solutions or suspensions, sterile non-parenteral solutions or oral solutions or suspensions, oil in water or water in oil emulsions and the like. Formulations or parenteral and nonparental drug delivery are known in the art and are set forth in Remington's Pharmaceutical Sciences, 19th Edition, Mack Publishing (1995). The dosage of the vaccine will depend inter alia on route of administration and will vary according to the species to be protected. One or more additional administrations may be provided as booster doses, usually at convenient intervals, such as two to three weeks.

In another embodiment, the method of use of the vaccine comprises isolating tumor cells or cancer cells from either donors or the patient to be treated, as described below. The isolated cancer cells are transfected with the above-described expression vector and are grown under conditions such that the co-stimulatory molecule and antigen are expressed. As a result of this arrangement, the cancer cells are effectively converted to antigen presenting cells (APC). The APC cancer cells are then exposed to T cells isolated from the patient, either in vivo or ex vivo. That is, in one embodiment, the APC cancer cells are irradiated to prevent reproduction of the APC cancer cells prior to injecting the APC cancer cells into the patient. In another embodiment, T cells isolated from the patient arc exposed to the APC cancer cells and are then isolated from the APC cancer cells before being injected into the patient. In both embodiments, the T cell response is activated which in turn elicits an immune response against tumors. Furthermore, once the tumor has been destroyed, memory cells remain, meaning that the patient is effectively immunized against the tumor, and a subsequent immune response will be faster and stronger.

Thus, as discussed above, the instant invention relates to the development of a new method of cancer immunotherapy and its in vitro, ex vivo, and in vivo uses. More specifically, this invention relates to the development of DNA vector comprising a co-stimulatory molecule and antigen and the protocol suitable for the in vitro generation of genetically modified human cancer cells for cancer therapy. These cells share phenotypes of both antigen presenting cells and cancer cells and are suitable as a cellular vaccine for certain types of cancer.

The present invention provides methods and compositions for use of genetically modified cancer cells to activate T cells for immunotherapeutic responses against primary or metastatic cancer. The cancer cells obtained from human donors, after transfection or transduction with the co-stimulator and the antigen expression vector described above, are administered to a cancer patient to activate the relevant T cell responses in vivo. Alternatively, T cells from patients are exposed to genetically modified cancer cells in vitro to activate the relevant T cell responses in vitro. The activated T cells are then administered to a cancer patient. In either case, the genetically modified cancer cells are advantageously used to elicit an immunotherapeutic growth-inhibiting response against a primary or metastatic tumor.

The invention is further illustrated by the following non-limited examples. All scientific and technical terms have the meanings as understood by one with ordinary skill in the art. The specific examples which follow illustrate the methods in which the compositions of the present invention may be prepared and are not to be construed as limiting the invention in sphere or scope. The methods may be adapted to variation in order to produce compositions embraced by this invention but not specifically disclosed. Further, variations of the methods to produce the same compositions in somewhat different fashion will be evident to one skilled in the art.

### EXAMPLES

The examples herein are meant to exemplify the various aspects of carrying out the invention and are not intended to limit the invention in any way.

### Example I

### Construction of Yeast Hybrid Vaccine Easy Delivery System Expression Vector Plasmids for IL-2 and a Universal Vector for Antigen Genes Derived From Disease Pathogen

The plasmid pcDNA3.1-L-2, (shown in FIG. 2) consisting of human interleukin-2 (IL-2) was a gift from Dr. He P at Shanghai Medical University in China. The IL-2 gene in pcDNA3.IL-2 plasmid was digested with restriction enzymes smalI/NotI and cloned into pYES3/CT (Invitrogen) with smalI/NotI in its MCS. The plasmid pYEX-BX (Clontech) was modified by introducing more restriction sites in its MCS for easy cloning as a universal vector, pYEX.UIG.

The resulting universal vector can be engineered to express antigen genes from disease-causing pathogen. In this case, a HIV-1 fusion antigen-1 (gp41, gp36) (a gift from Dr. Ho Min at AIDs research center, China) was cloned into the pYEX.UIG with EcoRI/Xho ligation.

### Example II

### Construction of Yeast Hybrid Vaccine Easy Delivery System

As shown in FIG. 2, the resulting plasmid pYES3/CT.IL-2 was transformed into fungi strain W303-1A a (ATCC) using TE/LiOAc method. The transformants were selected by plating the aliquots of the transformation on selective plates that containing Leu dropout auxotrophic agar media. The resulting plasmid pYEX.UIG.gp41 was transformed into W303-1A a (ATCC) with the same transformation method. The transformants were selected with URA3 drop out agar media plate. The selected two IGV.IL-2 (a) and IGV.gp41.36(α) were mated together by cross gene type mating. The mated vector with its inserts is able to express both the IL-2 and the antigen in independent fashion. This yeast hybrid gene expressing unit becomes the final vaccine product.

### Example III

hIL-2 Assay

One yeast vector was engineered with IL-2 as the co-stimulator, another yeast vector was engineered with HIV-1 fusion antigen gp41.36. Yeast transformation was carried out with TE/LiOAc method. Mating of the parent haploid yeasts was carried out with cross plating. 8 month female DBA/J mice were injected with 1 ml of mineral oil intraperitoneally. 24 hrs after the injection, macrophages were obtained from peritoneal exudates with serum-free Hanks' PBS(HPBS) washout. 1−6×10⁶ macrophages were aliquoted into two groups and incubated with IGV.gp41.36 and IGV.factor respectively. At indicated days. 200 ml aliquots were removed and stored at −70° C. for quantification. Secreted hIL-2 was quantitated using the hIL-2 DuoSet kit (Genzyme Diagnostics, Cambridge, Mass.).

### Example IV

### CTL Assays

Splenocytes were obtained from mice at each group and cocultured with 100 μCi of ⁵¹Cr labeled B16.gp41.36 cells at a varying E:T ratio. The percent specific lysis was calculated as experimental cpm subtracting spontaneous cpm.

### Example V

### Vaccination on Animal Model

4-6 week female C57B1/6 mice (H-2^{b}) from Jackson Laboratories (Bar Harbor, Me.) were injected with 1 million B16 melanoma stably transfected to express HIV-1.gp41.36 by S.C. Mice (10 per group) were vaccinated subcutaneously twice at day 7 and 14 with 2 mg of the vaccine product suspended in 200 μl of saline water. Control mice were vaccinated with the vector product with IL-2 but without HIVgp41.36 and saline water alone. On day 28, tumor volume was measured using a digital caliper.

### Example VI

### hIL-2 Expression

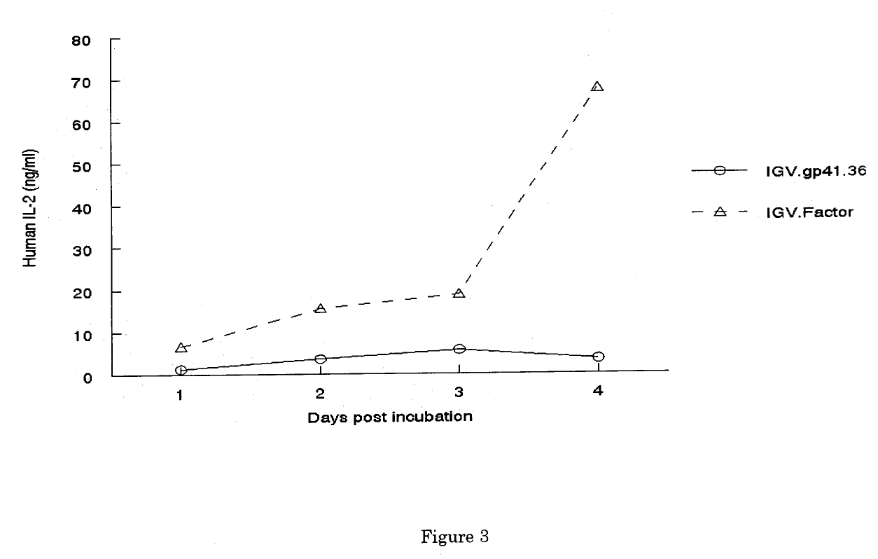

As shown in FIG. 3 the expression of hIL-2 levels derived from the product IGV.Factor that contains both IL-2 and gp41.36 inserts was between 6 and 67 ng/ml compared with hIL-2 levels from 1 and 3 ng/ml derived from IGV.gp41.36 without IL-2. The production of hIL-2 from IGV.gp41.36 and IGV.IL-2 stimulated macrophages. Cells were incubated at indicated days. Aliquots were removed, and supernatant were analyzed by ELISA for hIL-2.

### Example VII

### CTL Activity in Cured Mice

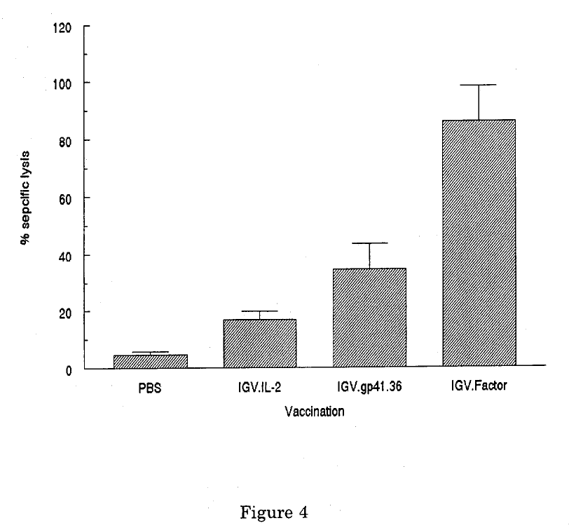

The mice were enthanized with CO₂ inhalation and their spleens were removed 120 days after challenge. Splenocytes were prepared IGV.Factor vaccinated mice demonstrated 85% specific lysis at an E:T ratio of 40:1 compared with other control vaccines IGV.gp41.36 with 34%, IGV.IL2 with 16% and PBS with 4%. FIG. 4 demonstrates the Cytotoxic T lymphocyte activity in mice vaccinated with indicated vaccines. Splenocytes were co-cultured with B16.gp41.36 cells for 5 days and then measured for CTL activity as described in materials and methods.

### Example VIII

### Generation of Systemic Protective Immunity

Mice vaccinated with IGV.Factor and other control vaccines after tumor reach the palpable size (normally 50-70 mm³) were monitored for tumor regression over 120 days. Complete protective immunity is defined as mouse remain tumor free for more than 21 days after tumor completely regression. Otherwise, it will be considered no protection. Mice treated with IGV.Factor demonstrated 93% protective immunity compared with other control vaccines at lower rates, see Table 1.

TABLE 1
Protective Immunity induced by IGV vaccines

| | Responses | | | |
|---|---|---|---|---|
| Vaccines | Experiment 1 | Experiment 2 | Experiment 3 | Total |
| IGV.Factor | 9/10 | 10/10 | 9/10 | 93% |
| IGV.gp41.36 | 6/10 | 4/10 | 5/10 | 50% |
| IGV.factor + | 2/10 | 4/10 | 3/10 | 30% |
| IL-2 | | | | |
| PBS | 0/10 | 0/10 | 0/10 | 0% |

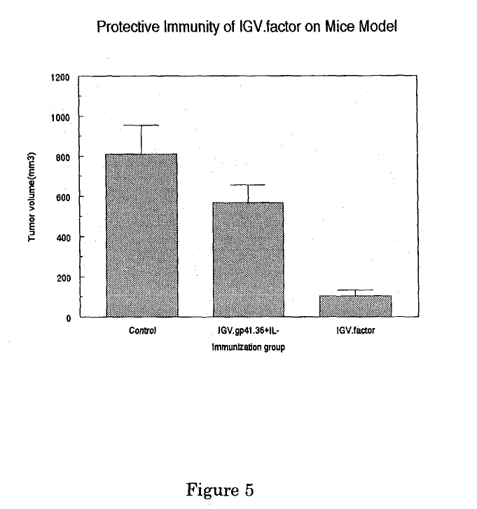

FIG. 5 shows mice treated with PBS as control, IGV.gp41.36+IL-2 and IGV.factor as immunization. Mice received PBS showed continues tumor growth compared with complete tumor regression in IGV.factor vaccinated mice. Mice treated with IGV.gp41.36 plus recombinant IL-2 shows protection, but no significant as compared with IGV.factor group. All of the experiments discussed above have been repeated more than three times with similar results. Surprisingly, the vaccine of the present invention demonstrated a 93% protective immunity compared with other control vaccines. This level of protection is rarely seen with vaccines.

New generation vaccines, particularly those based on recombinant proteins and DNA are less immunogenic than traditional vaccines. Therefore, there is an urgent need for the development of new and improved vaccine delivery systems. The current vaccine technologies, especially in inducing cell-mediated immune response, have focused on introducing antigen in vivo via various efficient delivery systems. All those technologies lack delivering the powerful partner factor in one delivery vector system but expressed in a genetically independent fashion.

The concept of this invention is that every antigen needs its perfect partner to co-stimulate the protective immunity. It is very likely that the antigen could be less reactogenic than with its co-stimulator delivered coincidently. This concept can be expanded to all gene therapy areas that every molecule or factor delivered in vivo may need its partner growth factor existed at an ideal ratio in order to achieve its maximum biological function.

This novel and innovative vaccine technology platform provides a broad range concept of developing both preventive and therapeutic diseases vaccines, especially in stimulating CTL mediated immune response.

The invention therefore encompasses the use of organisms carrying the expression vector 10 of the present invention produced according to the methods of the invention for any number of human or veterinary therapeutics. For example, vector types other than above mentioned combination of genes may be produced as described herein can be used in ex vivo cell transplantation therapies for the treatment of a variety of human diseases, e.g., disorders of the immune system. Accordingly, such cells are useful for modulating autoimmunity and limiting a variety of autoimmune diseases.

The subject vaccines and antimicrobial drugs may be used in a wide variety of vertebrates, and will find particular use with mammals, such as man, and domestic animals. Domestic animals include avian species, bovine, ovine, porcine, equine, caprine, leporidate e.g., rabbits, or other animals which may be held in captivity or may be a vector for a disease affecting a domestic vertebrate. Suitable individuals for administration include those who are, or suspected of being, at risk or exposure to bacteria, viral or other pathogenic diseases as well as those who have been exposed and/or infected.

Gene-modified cancer cells also find use in the ex vivo expansion of T cells, e.g., CD4⁺ cells or CD8⁺ cells or both. Thus, such cells are useful for stimulating the proliferation and reconstitution of CD4⁺ cells or CD8⁺ cells or both in a human having an immune disorder. Reconstitution of the immune system, e.g., a patient's CD4⁺ cells, is useful in immunotherapy for preventing, suppressing, or inhibiting a broad range of immunological disorders, e.g., as found during HIV infection.

The cancer cells described herein are also useful for vaccine development. For example, administration of antigens (as a form of cell lysate) to immuno-competent host further facilitate the use of these cells for active immunization in situ.

In addition, gene-modified cancer cells are useful for the generation of antibodies (e.g., monoclonal antibodies) that recognize cancer cell-specific markers. Anti-cancer cell antibodies are produced according to standard hybridoma technology. Such antibodies are useful for the evaluation and diagnosis of a variety of immunological disorders.

In some embodiments, the gene-delivery system as described herein at therapeutically effective concentrations or dosages may be combined with a pharmaceutically or pharmacologically acceptable carrier, excipient or diluent, either biodegradable or non-biodegradable. Exemplary examples of carriers include, but are by no means limited to, for example, poly(ethylene-vinyl acetate), copolymers of lactic acid and glycolic acid, poly(lactic acid), gelatin, collagen matrices, polysaccharides, poly(D,L lactide), poly(malic acid), poly(caprolactone), celluloses, albumin, starch, casein, dextran, polyesters, ethanol, mathacrylate, polyurethane, polyethylene, vinyl polymers, glycols, mixtures thereof and the like. Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars and starches. See, for example, Remington: The Science and Practice of Pharmacy, 2000, Gennaro, A R ed., Eaton, Pa.: Mack Publishing Co.

The invention provides kits for carrying out the methods of the invention. Accordingly, a variety of kits are provided. The kits may be used for any one or more of the following (and, accordingly, may contain instructions for any one or more of the following uses): use for therapeutically or prophylactially treating an individual against a pathogenic organism such as a viral, fungal or bacterial infection; treating some forms of cancer in an individual; preventing the spread or metastasis of some forms of cancer; preventing one or more symptoms of some forms of cancer; reducing severity of one or more symptoms associated with cancer; delaying development of cancer in an individual; or vaccinating an individual against some forms of cancer.

The kits of the invention comprise one or more containers comprising the gene-delivery system and a suitable excipient as described herein and a set of instructions, generally written instructions although electronic storage media (e.g., magnetic diskette or optical disk) containing instructions are also acceptable, relating to the use and dosage of the gene-delivery system for the intended treatment. The instructions included with the kit generally include information as to dosage, dosing schedule, and route of administration for the intended treatment. The containers of the gene-delivery system may be unit doses, bulk packages (e.g., multi-dose packages) or sub-unit doses.

The gene-delivery system may be packaged in any convenient, appropriate packaging.

As will be appreciated by one knowledgeable in the art, the vaccine may be combined or used in combination with other treatments known in the art.

The foregoing description is considered as illustrative only of the principles of the invention. Further, since numerous modifications and changes will readily occur to those skilled in the art, it is not desired to limit the invention to the exact construction and process shown as described above. Accordingly, all suitable modifications and equivalents may be resorted to falling within the scope of the invention as defined by the claims that follow. The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

The embodiments of the invention in which an exclusive property or privilege is claimed are defined as follows:

1. A vaccine for provoking an immunological response in a host to be vaccinated comprising:
a plasmid having a DNA molecule encoding a co-stimulator molecule operably linked to a promoter and at least one antigen operably linked to a promoter; and
an organism for carrying said plasimd.

2. The vaccine of claim 1, wherein the co-stimulatory molecule is selected from the group consisting of: IL-2, TNF, IFN, B7-1, GM-CSF, IL-12, IL-3, IL-4, IL-5, IL-6, IL-10, IL-12, IL-14, IL-14, IL-18, B7-2, CD28, CD40, TNFR, lymphotoxin-betaR, NF-KappaB, ICAM-1, LFA-3, M-CSFR, mM-CSF, Flt3-L, SCF, TPO, CD80, CD58, 6M-CSF, and B-7.

3. The vaccine of claim 1, wherein the co-stimulatory molecule is IL-2.

4. The vaccine of claim 1, wherein the co-stimulatory molecule is B7-1.

5. The expression system according to claim 1, wherein the antigen is derived from a viral, bacterial, fungal, helminth, ectoparasite, or cancer cell.

6. The vaccine of claim 1, wherein said host is a vertebrate.

7. The vaccine of claim 6, wherein said vertebrate is a mammal.

8. The vaccine of claim 6, wherein said vertebrate is a human.

9. The vaccine of claim 6, wherein said vertebrate is a domestic animal.

10. The vaccine of claim 6, wherein said vertebrate is a chicken.

11. The vaccine of claim 1, wherein said microorganism is prokaryotic.

12. The vaccine of claim 1, wherein said microorganism is eukaryotic.

13. The vaccine of claim 11, wherein said prokaryotic microorganism is a bacteria.

14. The vaccine of claim 11, wherein said prokaryotic microorganism is a virus.

15. The vaccine of claim 12, wherein said eukaryotic microorganism is fungus.

16. The vaccine of claim 15, wherein said fungus is a yeast.

17. A vaccine for provoking an immunological response in a host to be vaccinated comprising:
a microorganism that is attenuated as the result of at least one mutation in its genome and wherein said microorganism carries a plasmid capable of expressing a co-stimulator molecule and at least one antigen.

18. A vaccine used to vaccinate a host comprising a pharmaceutically acceptable excipient, and an organism carrying a plasmid capable of independently expressing a co-stimulator molecule and at least one antigen.

19. A method for preparing a microorganism capable of eliciting an immunological response by a host susceptible to disease comprising:
mating two separate haploid yeast strains, wherein one of said haploid yeast strains comprises a plasmid capable of expressing a co-stimulator molecule and the other haploid yeast strain comprises a plasmid capable of expressing at least one antigen.

20. The method of claim 19, wherein said host is a vertebrate.

21. The method of claim 19, wherein said vertebrate is a mammal.

22. The method of claim 21, wherein said mammal is a human.

23. The method of claim 21, wherein said mammal is a domestic animal.

24. The method of claim 20, wherein said vertebrate is a chicken.

25. The method of claim 19, wherein said desired antigen or antigens is/are chosen from the group comprising: viral, bacterial, protozoan, helminth, ectoparasite and cancer derived agents.

26. The method of claim 25, wherein said bacterial derived agents are Fragment C of tetanus toxin, the B subunit of cholera toxin, the hepatitis B surface antigen, Vibrio cholerae LPS, HIV antigens and/or Shigella soneii LPS.

27. The method of claim 25 wherein said cancer agents are K-ras, EGFR, HER2, PSMA, CEA, MAGE, MART-1.

28. A method of treating a host infected with a pathogenic microorganism comprising:
administering a to the host a composition comprising an organism carrying a plasmid capable of independently expressing a co-stimulator molecule and at least one antigen.

29. A method of eliciting an immune response in an individual comprising administering the immunogenic composition of claim 1 to the individual in an amount sufficient to elicit an immune response.

30. The method of claim 29, wherein the immune response persists more than about four weeks after administration.

31. An expression vector comprising a DNA molecule encoding a co-stimulator molecule operably linked to a promoter and at least one antigen operably linked to a promoter.

32. A cancerous cell transfected with the expression vector of claim 31.
